(19) United States
(12) Patent Application Publication
(10) Pub. No.: US 2009/0069695 A1
(43) Pub. Date: Mar. 12, 2009

(54) DEVICE FOR IMAGING A TURBID MEDIUM

(75) Inventors: Tim Nielsen, Hamburg (DE); Dirk Grosenick, Schildow (DE); Herbert Heinrich Rinneberg, Berlin (DE); Oliver Heinz Steinkellner, Berlin (DE)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/293,109

(22) PCT Filed: Mar. 12, 2007
(86) PCT No.: PCT/IB07/50804

§ 371 Date: Sep. 16, 2008

(30) Foreign Application Priority Data
Mar. 17, 2006 (EP) 06111321.3

(51) Int. Cl. G01N 21/49; A61B 6/00

(52) U.S. Cl. 600/473; 250/341.1

(57) ABSTRACT

The present invention relates to a device for imaging a turbid medium (130, 132; 184) comprising: means (110; 134, 138, 140, 142, 144, 146) for optically scanning a predefined maximum area of a scanning plane (102; 104) for acquisition of imaging data, means (134, 136; 206, 208, 210) for detection of an outer contour of the turbid medium, means (112, 120, 122) for controlling the optical scanning such that a sub-area of the maximum area is scanned that is smaller than the maximum area and that covers the outer contour.

### FIELD OF THE INVENTION

The present invention relates to the field of optical imaging, and more particularly without limitation to optical mammography.

### BACKGROUND AND RELATED ART

U.S. Pat. No. 6,718,195 B2 and U.S. Pat. No. 6,922,582 B2 show methods and devices for localizing a deviant region in a turbid medium by means of optical imaging.

These methods can be used in optical mammography where a breast of a female body is examined using light. Said methods produce images in which any deviations, for example tumors, can be clearly recognized. This is achieved inter alia by providing markers in an image of the turbid medium.

A method and a device of this kind are known from “Clinical Optical Tomography and NIR Spectroscopy for Breast Cancer Detection”, S. B. Colak et al, IEEE Journal of Selected Tops in Quantum Electronics, Vol. 5, No. 4, July/August 1999. The known method and device are used for imaging the interior of biological tissues. The method and the device can be used inter alia in medical diagnostics for in vivo breast examinations for visual localization of any tumors present in the breast tissue of a human or animal female body. According to the known method a turbid medium is successively irradiated by light from various irradiation positions. Subsequently, the intensity of the light having been transported along different light paths through the turbid medium that extend from their irradiation position is measured in a number of measuring positions. The intensities measured are used for the reconstruction of an image of the turbid medium. A spatial distribution of the attenuation of the light through the tissue is reproduced in this image. Light is attenuated by tissue in that the tissue scatters and absorbs the light.

Similar techniques are disclosed in “Time-domain scanning optical mammography: I. Recording and assessment of mammograms of 154 patients”. D. Grosenick et al., Phys. Med. Biol. 50 (2005) 2429-2449, in particular section 7, pp. 2443-2446 and “Diffuse optical tomography and spectroscopy of breast cancer and fetal brain” , Regine Choe, Dissertation, University of Pennsylvania, 2005.

### SUMMARY OF THE INVENTION

In accordance with the present invention there is provided a device for imaging a turbid medium comprising means for optically scanning a predefined maximum area of a scanning plane for acquisition of imaging data, means for detection of an outer contour of the turbid medium, and means for controlling the optical scanning such that a sub-area of the maximum area is scanned that is smaller than the maximum area and that covers the outer contour.

The means for optically scanning may be implemented by an optical scan system, the means for detection of the outer contour may be implemented by a detection system, and the means for controlling may be implemented by a control system.

The optical scan system has a suitable light source or can be coupled to such a light source. Further, the optical scan system has one or more detectors for detection of transmitted and/or return radiation and/or it can be coupled to such a detector or detectors. The optical scan system may or may not have separate detectors located at the source and target sides of the optical scan system.

The optical scanning is performed using light, such as laser light. The term ‘light’ is to be understood in the context of the present invention to mean electromagnetic radiation of a wavelength in the visible or infrared range between approximately 400 and 1400 nm. The turbid medium is to be understood to mean a substance consisting of a highly light scattering material. More specifically, in the context of the present invention the term turbid medium is to be understood to mean biological tissue. A deviant region is to be understood to mean a region in which the turbid medium deviates in any way or form from the turbid medium in the surrounding region. More specifically, in the context of the present invention such an area is to be understood to mean a region comprising tumor tissue.

The present invention is particularly advantageous as it does not require use of X-radiation for image acquisition. Moreover the present invention solves the technical problem of reducing the image acquisition time. Embodiments of the invention solve this and/or other technical problems, such as improving sharpness and/or spatial resolution of the acquired images.

It is to be noted that the present invention purely relates to imaging but not to treatment of the human body or to diagnosis.

Embodiments of the present invention are particularly advantageous as knowledge of the outer contour of the breast greatly facilitates reconstruction of the absorption and scattering properties of the breast tissue as well as reconstruction of the concentration of a fluorescent contrast agent. Furthermore, the image acquisition time can be reduced substantially by limiting the optical scanning to a sub-area of a maximum scanable area. This is due to the fact that an outer contour of the turbid medium is detected before the optical scanning is performed such that the optical scanning can be limited to a sub-area that still covers the turbid medium but reduces coverage of areas outside the turbid medium that are not of interest for the image acquisition. Reduction of the image acquisition time is a substantial advantage for dynamic contrast agent studies as well as easing the problem of patient movement, such as due to breathing, during the data acquisition and thus leads to sharper images.

Moreover, decreasing the data acquisition time is particularly advantageous for dynamic measurements, such as for imaging a wash-in and/or wash-out process of a contrast agent. The reduction of the data acquisition time enables to acquire more images during the wash-in and/or wash-out periods.

In accordance with an embodiment of the invention a moveable optical fiber is used for performing the optical scanning. The optical fiber is moved into the scanning positions such as by means of an xy-stepper motor. The moveable optical fiber can be carried by a measurement head. In addition to the optical fiber that is used for irradiating the turbid medium, the measurement head can carry a plurality of optical fibers for detection of return radiation.

In accordance with an embodiment of the invention a fixed light source and a controllable mirror is used for performing the optical scanning. For example, a so called galvano mirror is used as such a controllable mirror for performing the optical scanning. This facilitates usage of a charge coupled device (CCD) sensor array for detection of the return radiation that is returned from the turbid medium in response to the optical scanning in the reverse direction. This is particularly advantageous as the CCD sensor array does not need to be moveable.

In accordance with an embodiment of the invention the return radiation that is returned from the turbid medium in a reverse direction is detected at various positions. The detector that is located on the source side may be designed to cover various detection positions that have different distances from the source. These detection positions can be implemented in a measurement head, by a CCD sensor array or otherwise. This enables to detect return radiation that has traveled along various paths through the turbid medium before reaching one of the detectors on the source side.

In accordance with an embodiment of the invention continuous wave light or trains of sub-nanosecond light pulses are used for performing the optical scanning. In the latter case the pulse shapes of the return radiation and/or of the transmitted radiation that is received in response to the optical scanning are acquired. The pulse shape information is used as imaging data as it contains information as to reflection and absorption properties of the turbid medium along the respective photon trajectories.

In accordance with an embodiment of the invention the optical scanning is performed from two directions, for example from two opposite directions, in order to cover a larger number of different light paths through the turbid medium. For this purpose at least one of the components of the optical scanner can be rotatably mounted with respect to the source and target plates in order to vary the direction from which the optical scanning is performed.

In accordance with an embodiment of the invention primary and secondary radiation that is returned from or transmitted through the turbid medium is detected. The primary radiation directly results from the light with which the turbid medium is irradiated during the optical scanning process. Hence, the primary radiation is due to scattering and absorption within the turbid medium. Primary radiation is received as primary return radiation at the source side and as primary transmitted radiation at the target side. The secondary radiation is due to photon emissions of the turbid medium, such as by fluorescence, that is excited by the incident source light beam, e.g. after administration of a fluorescent agent. Hence, the secondary radiation can have a different frequency than the primary radiation. Secondary radiation can also be detected at the source side (“secondary return radiation”) and/or at the target side (“secondary transmitted radiation”).

Embodiments of the invention facilitate the time-resolved detection of the primary and secondary radiations, such as of laser light and induced fluorescence light. Primary and secondary radiation is detected both on the target and the source side to enhance the spatial resolution in the projection direction. The diffusely reflected incident light of the light source, such as laser light, and the fluorescence light contain information on the depth of a structure in the turbid medium, such as a tumor, because signals from different depths arrive at different times at the detectors and have different temporal shape.

In accordance with an embodiment of the invention multiple detectors with different distances to the light source are used as the maximum sensitivity of the measurement towards the tissue volume sampled by the detected light is approximately a curved shape with a maximal depth corresponding to about half the spacing between the source and detector planes. Using multiple detectors allows to cover a range of depths simultaneously.

In accordance with an embodiment of the invention the device is symmetric with respect to the mid-plane between the source and target plates in order to achieve maximum sensitivity.

In another aspect the present invention relates to an imaging device and an imaging method comprising means for optical scanning, such as a scanning system, wherein continuous wave or pulsed radiation is used for performing the optical scanning and further comprising means (124) for a time resolved acquisition of shapes of waves and/or pulses of primary and/or secondary radiation. This may be used independently of or in combination with the above-described acquisition of the outer contour of the turbid medium for reduction of the scanning area.

In another aspect the present invention relates to an imaging device and an imaging method comprising means for optical scanning, such as a scanning system, and mechanical means for gently compressing the turbid medium, such as a female breast, located between the source and target plates in order to reduce the thickness of the turbid medium in the source-target direction. This has the advantage that the intensity of the transmitted radiation and thus the signal-noise ratio can be substantially improved. This may or may not be used in combination with the above described acquisition of the outer contour of the turbid medium for reduction of the scanning area.

### BRIEF DESCRIPTION OF THE DRAWINGS

In the following embodiments of the invention are explained in greater detail, by way of example only, making reference to the drawings in which:

FIG. 1A is a block diagram of an embodiment of an imaging device of the invention,

FIG. 1B shows exemplary pulse shapes of transmitted and return radiation,

FIG. 2 is a schematic cross sectional view of an alternative embodiment of an imaging device of the invention,

FIG. 3 is a schematic top view of a measurement head for use at the source side,

FIG. 4 is a schematic top view of a measurement head for use at the target side,

FIG. 5 is a schematic cross sectional view illustrating a number of different photon trajectories through a turbid medium,

FIG. 6 shows a first embodiment of a detector for sequential detection of primary and secondary radiation,

FIG. 7 illustrates an embodiment of a detector for concurrent detection of primary and secondary radiation,

FIG. 8 is a block diagram of an embodiment of the imaging device of the invention using a CCD sensor array as a detector,

FIG. 9 is a flowchart illustrating a first embodiment of a method of the invention,

FIG. 10 is a flowchart illustrating the second embodiment of a method of the invention.

### DETAILED DESCRIPTION

Like elements shown in the various embodiments of the invention that have corresponding functionalities are designated by the same reference numerals throughout the following detailed description.

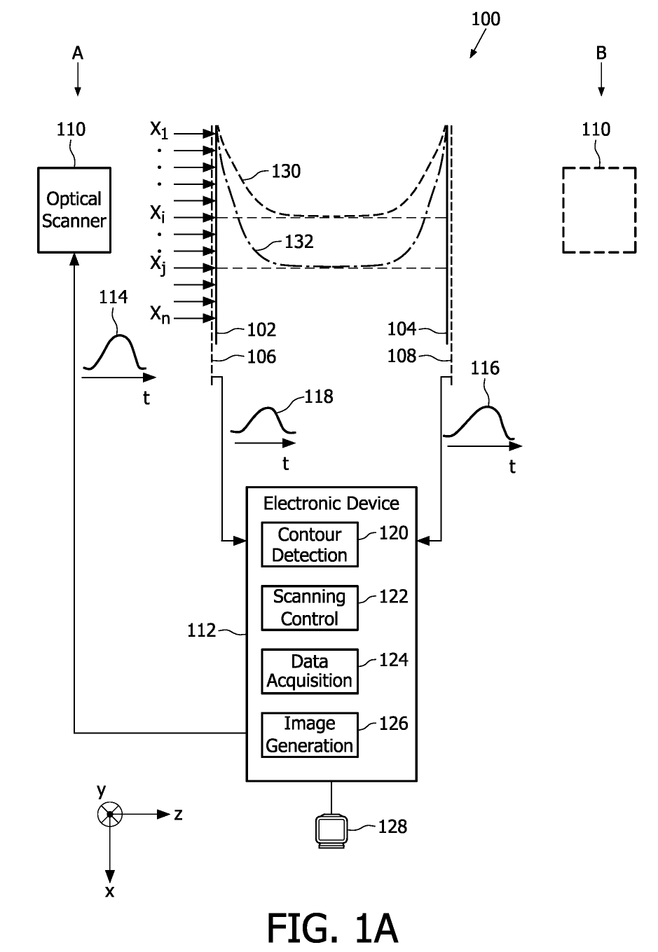

FIG. 1 shows an imaging device 100 for optical imaging of biological tissue, such as for optical mammography.

The imaging device 100 has a source plate 102 and a target place 104. The source plate 102 and the target plate 104 enclose a space for receiving the turbid medium to be imaged, such as a woman's breast. In the embodiment considered here the source plate 102 and the target plate 104 are substantially parallel and extend into the vertical direction. Alternatively the source and target plates 102, 104 can also be oriented otherwise, such as horizontally. The distance between the plates 102 and 104 can be adjustable. This facilitates to gently compress the breast between the plates 102, 104 while sufficient comfort for the woman is preserved. A gentle compression of the breast between the plates has the advantage of higher intensity of transmitted primary and secondary radiation, and that the breast is less likely to be inadvertently moved, e.g. due to breathing or other movements of the patient.

The source plate 102 defines a source plane 106 and the target plate 104 defines a target plane 108.

The imaging device 100 has an optical scanner 110 that comprises one or more light sources, such as laser light sources of various frequencies. The optical scanner is located on the source side of the imaging device 100.

The optical scanner 110 is coupled to an electronic device 112 that controls the image data acquisition process. The electronic device 112 can be a computer system, such as a personal computer, or a specialized electronic system.

The optical scanner 110 can be controlled by the electronic device 112 such that radiation reaches the source plane 106 at any one of the predefined scanning positions of which the scanning positions X₁ to Xₙ are shown in FIG. 1 by way of example. The optical scanner 110 is controlled by the electronic device 112 for scanning in the xy-plane.

The maximum scanable area is from the scan position X₁ to the scan position Xₙ as illustrated in FIG. 1. Likewise, there are maximum Y scan positions in the source plane 102 that are not shown in FIG. 1. For ease of explanation and without limitation of generality the following explanations refer to the X-direction only.

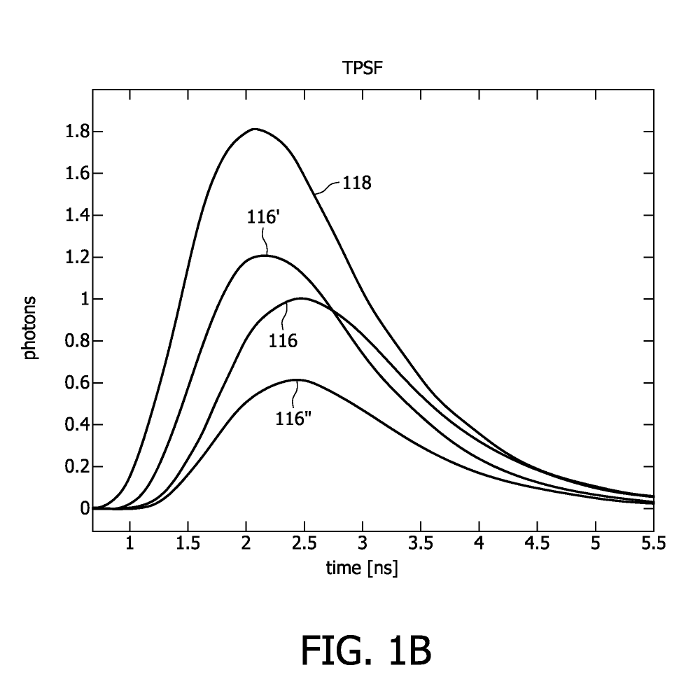

The imaging device 100 has a detector on both the source and target sides. The source detector serves for detection of primary and/or secondary return radiation that is returned from the turbid medium into a reverse direction whereas the target detector is used for detection of primary and/or secondary transmitted radiation that has traveled along a photon trajectory from the source to the target side. Embodiments of source and target detectors will be explained in greater detail with respect to the embodiments of FIGS. 2-8.

The electronic device 112 is coupled to both the target and source detectors for data acquisition of return and transmitted radiation that reaches the source plane 106 and the target plane 108, respectively.

Preferably, trains of sub-nanosecond light pulses are used for performing the optical scanning. FIG. 1 schematically shows a light pulse 114 provided by the light source of the optical scanner 110; the light pulse 114 reaches the source plane 106 at one of the scanning positions Xᵢ.

Due to scattering the light pulse 114 becomes substantially longer when it travels through the turbid medium between the source and target plates 102, 104. Further, the shape of the light pulse 114 is modified depending on the various photon trajectories contributing to the detected light pulse.

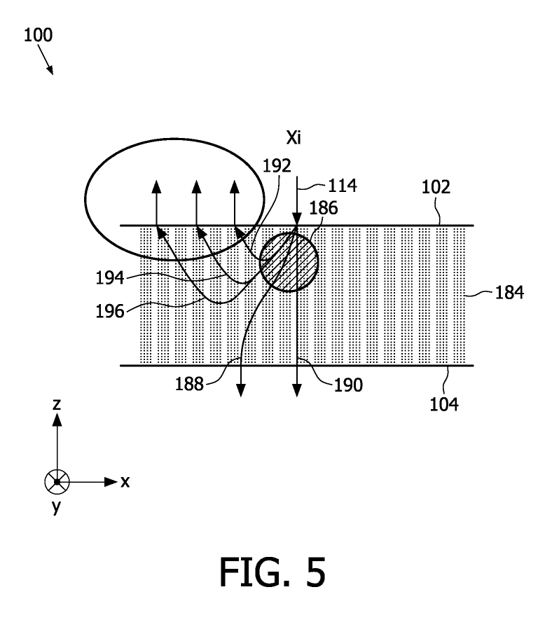

For example, a light pulse 116 is detected at the target plane 108 in response to the light pulse 114. Another light pulse 118 is detected at the source plane 106 also in response to the light pulse 114. The light pulses 116 and 118 that are returned from the turbid medium located between the target plate 104 and 106 have different shapes and lengths as they are due to different photon trajectories through the turbid medium as it will be explained in more detail with respect to FIG. 5.

The electronic device 112 has a module 120 for detecting an outer contour of the turbid medium that is located between source plate 102 and the target plate 104. The contour detection can be performed using signals provided from the source detector and/or target detectors. For example, if one of the source or target detectors is implemented using a CCD sensor array a picture can be taken from the turbid medium for acquisition of its contour.

Further, the electronic device 112 has a module 122 for controlling the optical scanner 110. The scanning control 122 is performed using the detected outer contour in order to exclude regions from the optical scanning process that are not of interest for imaging the turbid medium.

The electronic device 112 has a data acquisition module 124 for receiving and analyzing the signals provided by the source and target detectors, such as light pulses 116 and 118. The module 126 serves for generation of an image using the acquired data.

Depending on the implementation and/or selected operational mode the module 126 can produce separate images for radiation detected at the target and source sides and/or separate images for primary and secondary radiation. Alternatively the module 126 can combine data acquired at the target and source sides and/or primary and secondary return radiation into a single image.

The electronic device 112 is coupled to a monitor 128 for display of the resultant image.

It is to be noted that the various modules of the electronic device 112 can be implemented within the same or different physical units that can be tightly or loosely coupled. In particular, the functionalities of the electronic device can be implemented by a number of interoperable devices that are interoperable and coupled e.g. by means of a network.

In the following application of the imaging device 100 for optical mammography is considered. In operation a first woman's breast 130 is positioned between the source plate 102 and the target plate 104. Next, the outer contour of the breast 130 is detected. The outer contour of the breast 130 can be acquired by a projection of the breast 130 into the xy plane. This can be done by taking an image of the breast 130 using the source and/or target detector or a separate camera.

The image data that is acquired from the breast 130, e.g. by taking a picture, is entered into the module 120 in order to perform the detection of the outer contour of the breast in the xy plane. The outer contour of the breast's 130 projection in the xy plane provides a delimitation line for defining a sub-area within the maximum scannable area.

The optical scanning process can be limited to that sub-area as the scanning only needs to be performed where breast tissue of the breast 130 is located between the source plate 102 and target plate 104. In other words, if there is no breast tissue of the breast 130 at a scanning position Xₐ, Yₐ along the z direction that scanning position is outside the outer contour such that this scanning position does not need to be scanned.

Accordingly, the module 122 controls the optical scanner 110 such that the xy plane is only scanned at scanning positions of interest. This enables to substantially reduce the time required for performing the data acquisition especially for smaller breasts. This is particularly advantageous as a reduction of the data acquisition time increases patient comfort. Further, a reduction of the data acquisition time leads to sharper images as the patient is less likely to move, such as by breathing or otherwise, during a shorter image data acquisition time.

Moreover, decreasing the data acquisition time is particularly advantageous for dynamic measurements, such as for imaging a wash-in and/or wash-out process of a contrast agent. The reduction of the data acquisition time improves time resolution for such measurements and enables to acquire more images during the wash-in and/or wash-out periods.

During the optical scanning data is acquired from the source and target detectors and processed by the module 124 of the electronic device 112. The module 126 generates one or more images based on the acquired data. This may encompass data acquired by both the source and target detectors including pulse shape information of target and source light pulses (cf. light pulse 116 and 118) as well as fluorescence light pulses.

If target and source detectors are used that can operate on two frequencies concurrently, this enables to perform the data acquisition for both the primary radiation and the secondary radiation at the same time. If this is not the case two data acquisitions are performed sequentially for detection of the primary and the secondary radiations.

Preferably, the optical scanner 110 is rotatably mounted such that it can be moved from its position A as shown in FIG. 1 to an alternative position B as shown with dashed lines in FIG. 1. When the optical scanner has been moved to the position B the target side becomes the source side and vice versa.

It is advantageous to perform the optical scans from two opposite directions. Performing the optical scans from two opposite directions has the advantage that the spatial resolution in the z-direction can be increased as it will be explained in more detail with respect to FIG. 5.

As illustrated in FIG. 1 the sub-area for performing the optical scan for the breast 130 is limited between the X₁ and the Xᵢ positions. If a larger breast 132 is to be imaged the sub-area for optically scanning that breast 132 is limited between the X₁ and the Xj positions, where j>i, as the breast 132 is larger than breast 130.

It is to be noted that a fixed light source and a moveable mirror, such as a galvano mirror, can be used as an alternative to a moveable measurement head. This facilitates implementation of the source detector by a CCD camera.

Further, it is to be noted that it is advantageous to fill the space between the source and target plates 102 and 104 with a scattering fluid.

FIG. 1A shows exemplary pulse shapes of the light pulses 116 and 118 in the time domain. The light pulse 118 reaches its peak value more quickly than the light pulses 116, 116′ and 116″ as the photon trajectories that contribute to this light pulse peak 118 are on average shorter than those of the transmitted light pulses 116, 116′ and 116″.

Light pulse 116 is acquired for a scanning location without a lesion. Light pulses 116′ and 116″ are acquired for different lesions. FIG. 1A illustrates the impact of the respective lesions on the pulse shapes.

FIG. 2 shows an embodiment of the imaging device 100 that has a source measurement head 134 and a target measurement head 136. The source measurement head 134 has an optical fiber 138 for coupling to laser sources 140, 142, 144, 146, . . . ; each of the laser sources 140, 142, 144, 146, . . . can have a different frequency.

The measurement head 134 further comprises optical fibers 148, 150, 152, . . . that are coupled to respective detectors 154, 156, 158, . . .

The optical fibers 148-152 have different distances from the optical fiber 138 in order to cover different photon trajectories as it will be explained in more detail with respect to FIG. 5.

The laser sources 140, 142, 144, 146, . . . are selectable and controllable by the electronic device 112. The outputs of the detectors 154, 156, 158, . . . are coupled to the electronic device 112 for performing the data acquisition with respect to the source plane 106.

The target measurement head 136 has a number of optical fibers 160-168 that are coupled to respective detectors 170, 172, 174, 176, . . .

The outputs of these detectors 170-176 are also coupled to the electronic device 112 for performing the data acquisition with respect to the target plane 108.

Both of the measurement heads 134 and 136 are moveable in the xy directions on the source plane 106 and on the target plane 108, respectively. For example, both measurements heads 134, 136 are coupled with respective stepper motors that are controlled by the electronic device 112.

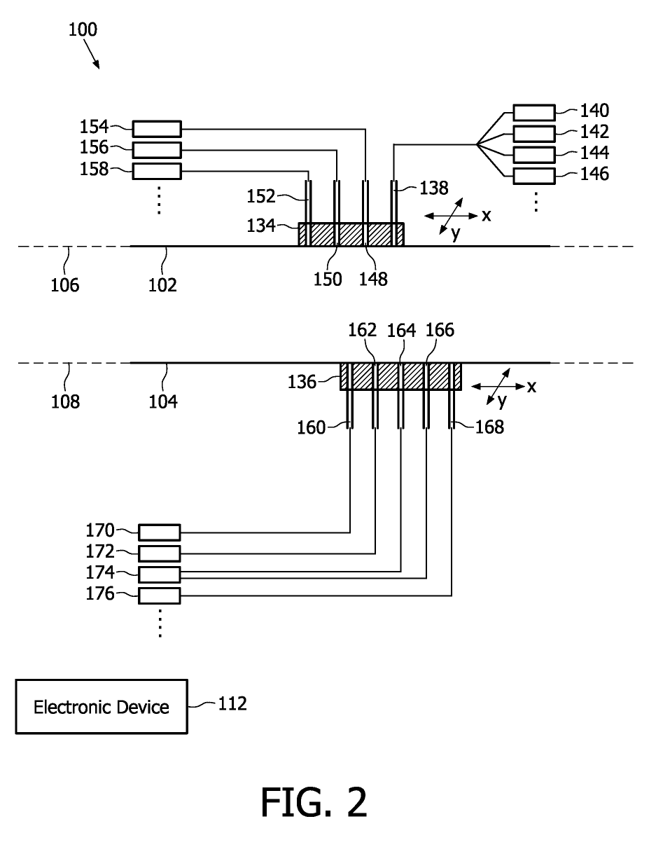

FIG. 3 shows a schematic top view of the measurement head 134 of FIG. 2. Other, e.g. two-dimensional arrangements are also possible. It is to be noted that the optical fibers 148-152 that are used for detection of the return radiation that is returned into the reverse direction have different distances 178, 180 and 182, respectively, to the optical fiber 138 that guides the radiation from one of the laser sources to the scanning position for coverage of different photon trajectories as shown in FIG. 5 below.

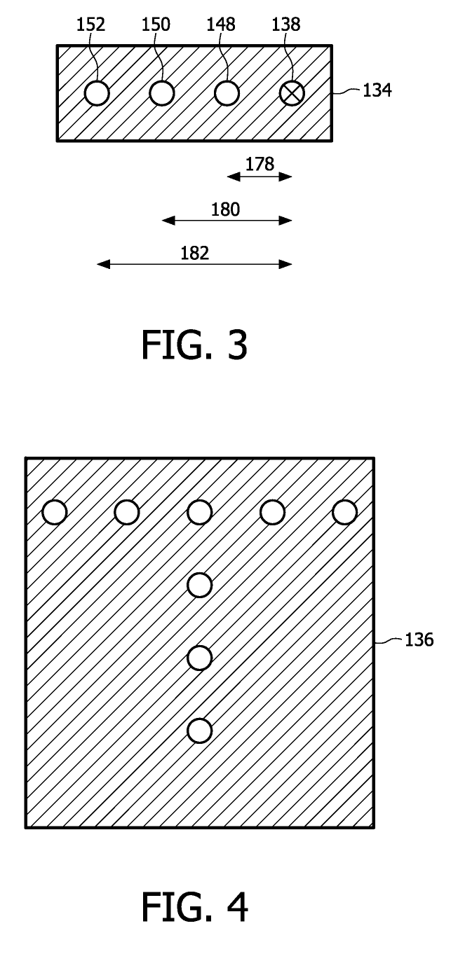

FIG. 4 shows a schematic top view of the measurement head 136 that is used for the target side as shown in FIG. 2. It is to be noted that the optical fibers of the measurement head 136 are arranged in a T-shape. While a T-shape is preferred other geometries for arranging the optical fibers are also possible.

FIG. 5 schematically illustrates a turbid medium 184, such as breast 130 or 132 (cf. FIG. 1), that is located between the source plate 102 and 104. The turbid medium 184 has a deviant region 186, such as a tumor, that has other light scattering, absorption and fluorescent dye uptake parameters than the rest of the turbid medium 184. FIG. 5 illustrates several average photon trajectories when the turbid medium 184 is irradiated with the light pulse 114 (cf. FIG. 1) at one of the scanning positions Xᵢ. The light pulse 114 results in various ensembles of photon trajectories that originate from the scanning position Xᵢ and terminate at a particular detector position where the photon trajectories 188 and 190 that extend from the source side to the target side of the imaging device 100 represent average trajectories. The respective light pulses that are transmitted along the average photon trajectories 188 and 190 are received by the target detector, such as the measurement head 136 (cf. FIG. 2 and light pulse 116 of FIG. 1).

Further, the light pulse 114 causes return radiation that is transmitted through the turbid medium 184 along average photon trajectories 192, 194, 196. These average photon trajectories end at the source plate 102 such that return radiation is received also in the reverse direction, i.e. opposite to the source-target direction. This return radiation can be detected e.g. by means of the measurement head 134 as depicted in FIG. 2.

The light pulses of the return radiation received via these average photon trajectories 188-194 have different lengths and shapes and arrive at different times due to the different average lengths of the photon trajectories and the different volumes of the turbid medium 184 covered by the photon trajectories.

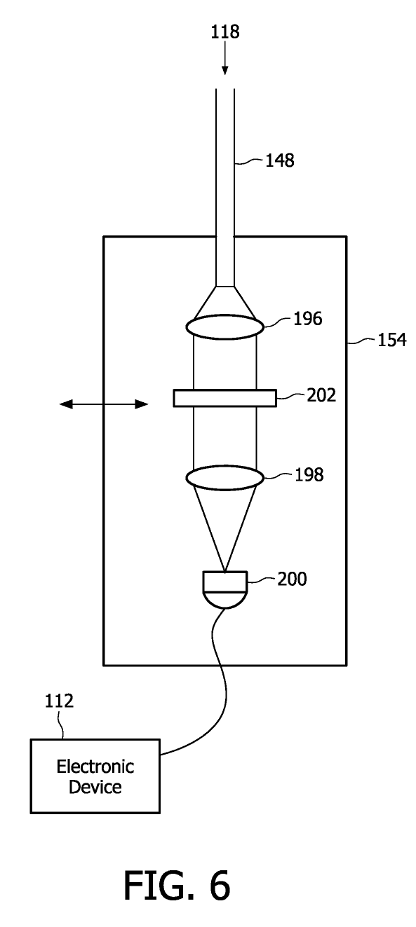

FIG. 6 shows an embodiment of one of the detectors that can be used for the source and/or the target side of the imaging device 100 (cf. detectors 154, 156, 158, . . . , 170, 172, 174, 176, . . . ). In the following an embodiment for the detector 154 is considered without restriction of generality. The detector 154 has a first optical lens 196 that is coupled to the optical fiber 148 as it is also shown in FIG. 2. The lens 196 is opposite to the lens 198 that focuses the light pulse 118 (cf. FIG. 1) onto a photodiode or photomultiplier 200. The output of the photomultiplier 200 is connected to the electronic device 112 (cf. FIG. 1 and FIG. 2).

An optical filter 202 can be inserted between the lenses 196 and 198. The filter 202 transmits radiation within a certain frequency range. For example, the frequency range is selected such that it allows transmission of secondary radiation but not of primary radiation.

For example, if a laser source is used for the primary radiation, the primary radiation is filtered out by the filter 202 whereas secondary radiation, such as radiation that is due to fluorescence, is transmitted such that it is detected by the photo multiplier 200. Hence, the embodiment of the detector 154 shown in FIG. 6 is useful for sequentially performing data acquisitions for primary and secondary return radiation.

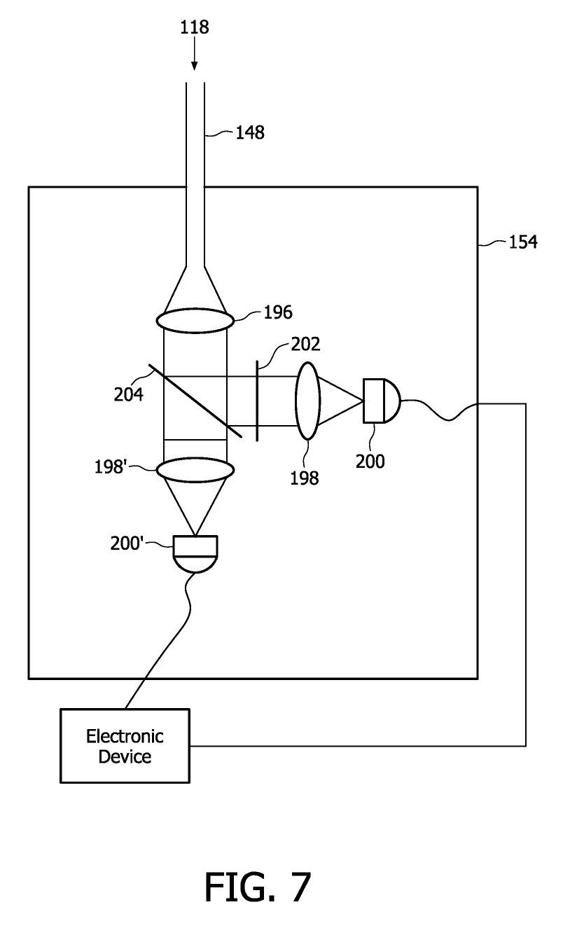

FIG. 7 shows an alternative embodiment for concurrent acquisition of primary and secondary return radiation. In this embodiment a beam splitter 204 is located in the light path between the lens 196 and its opposing lens 198′.

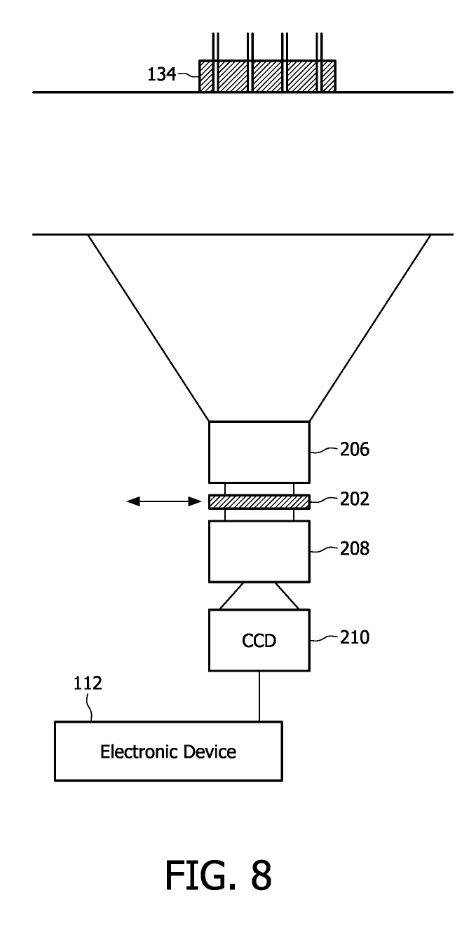

FIG. 8 illustrates an alternative embodiment for a target detector. The detector is equipped with an imaging optics with an optional filter, e.g. objective 206, optional filter 202, another objective 208 and a CCD sensor array 210. The objectives 206, 208 and the CCD sensor array 210 constitute a CCD camera that is coupled to the electronic device 112 for performing the data acquisition at the target site. Use of a CCD camera rather than a measurement head (cf. measurement head 136 of FIG. 2) allows for parallel data acquisition at a large number of detector positions at reduced costs and without movement. Another advantage of using a CCD camera is that it can be used for taking the picture of the turbid medium for acquisition of the outer contour.

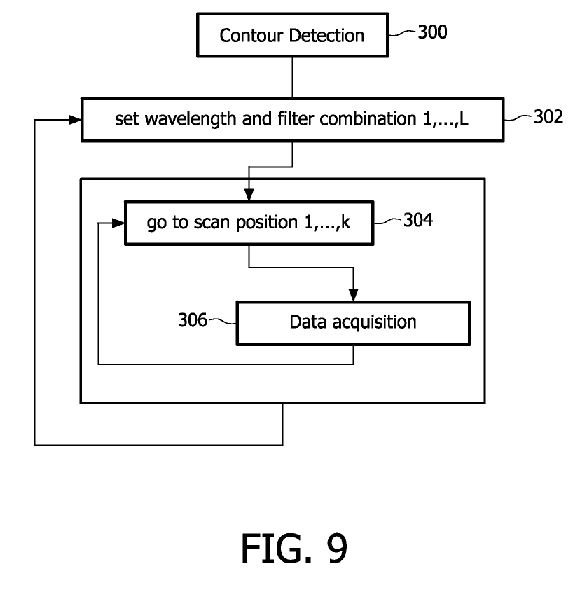

FIG. 9 shows a respective flowchart. In step 300 an outer contour of the turbid medium to be imaged is detected. The outer contour is used as a delimitation line for performing the optical scanning. For example, for imaging of the breast 130 shown in FIG. 1 the outer contour is detected such that the maximum X coordinate is Xᵢ.

In step 302 the wavelength and/or filter combination for performing the data acquisition are set.

In step 304 the optical scanner is controlled to scan all positions within the sub-area that covers the outer contour. In the X-direction that means that positions X₁ to X_{k=I} are scanned. At each scan position a data acquisition step 306 is performed.

After completion of the optical scan another wavelength and/or filter combination can be set in step 302 for performing a consecutive scan, such as for detection of fluorescence.

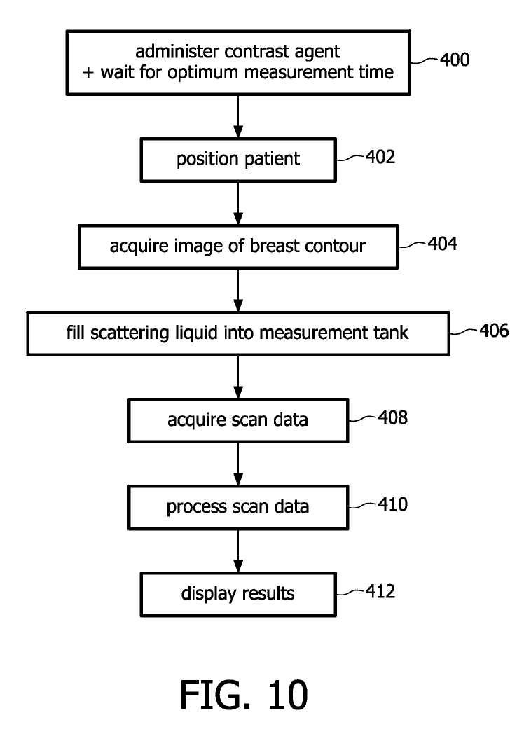

FIG. 10 shows an alternative embodiment of a method of the invention. In step 400 a fluorescent contrast agent is administered. After some time that is sufficient for distribution of the contrast agent within the patient's body, the patient is positioned in step 402 such as by positioning the patient's breasts between the source and target planes (cf. FIG. 1). In step 404 an image of the breast contour is acquired for detecting the outer contour of the breast, i.e. the breast's projection into the xy plane. This step corresponds to step 300 in the embodiment of FIG. 9.

In step 406 scattering liquid is filled into the measurement tank. In other words, a scattering liquid that has optical properties similar to the turbid medium is filled into the space enclosed between the target and the source plates. This simplifies the image generation algorithm for generating an image on the basis of the acquired data, as it is as such known from the prior art, namely the Choe reference cited above.

In step 408 the data acquisition is performed; this is analogous to steps 304 and 306 in the embodiment of FIG. 9.

In step 410 the acquired data is processed for generating one or more images. In step 412 the results are displayed.

### LIST OF REFERENCE NUMERALS

| | |
|---|---|
| 100 | Imaging device |
| 102 | Source plate |
| 104 | Target plate |
| 106 | Source plane |
| 108 | Target plane |
| 110 | Optical scanner |
| 112 | Electronic device |
| 114 | Light pulse |
| 116 | Light pulse |
| 118 | Light pulse |
| 120 | Module |
| 122 | Module |
| 124 | Module |
| 126 | Module |
| 128 | Monitor |
| 130 | Breast |
| 132 | Breast |
| 134 | Measurement head |
| 136 | Measurement head |
| 138 | Optical fiber |
| 140 | Laser source |
| 142 | Laser source |
| 144 | Laser source |
| 146 | Laser source |
| 148 | Optical fiber |
| 150 | Optical fiber |
| 152 | Optical fiber |
| 154 | Detector |
| 156 | Detector |
| 158 | Detector |
| 160 | Optical fiber |
| 162 | Optical fiber |
| 164 | Optical fiber |
| 166 | Optical fiber |
| 168 | Optical fiber |
| 170 | Detector |
| 172 | Detector |
| 174 | Detector |
| 176 | Detector |
| 178 | Distance |
| 180 | Distance |
| 182 | Distance |
| 184 | Turbid medium |
| 186 | Deviant region |
| 188 | Photon trajectory |
| 190 | Photon trajectory |
| 194 | Lens |
| 196 | Lens |
| 198 | Lens |
| 200 | Photomultiplier |
| 202 | Filter |
| 204 | Beam splitter |

1. A device for imaging a turbid medium (130, 132; 184) comprising:
means (110; 134, 138, 140, 142, 144, 146) for optically scanning a predefined maximum area of a scanning plane (102; 104) for acquisition of imaging data,
means (134, 136; 206, 208, 210) for detection of an outer contour of the turbid medium,
means (112, 120, 122) for controlling the optical scanning such that a sub-area of the maximum area is scanned that is smaller than the maximum area and that covers the outer contour.

2. The device of claim 1, the means for optically scanning comprising a moveable optical fiber (134) and means for moving the optical fiber for performing the optical scanning.

3. The device of claim 1, the means for optically scanning comprising a controllable mirror.

4. The device of claim 1, further comprising means (148, 150, 152, 154, 156, 158) for detecting return radiation that is returned from the turbid medium in response to the optical scanning.

5. The device of claim 4, the means for detecting the return radiation comprising a plurality of detectors (154, 156, 158) for detection of the return radiation at a plurality of positions.

6. The device of claim 1, further comprising a moveable head (134) for carrying the first optical fiber for irradiating the turbid medium and second optical fibers (148, 150, 152) for detection of return radiation that is returned from the turbid medium in response to the irradiation.

7. The device of claim 4, wherein the means for detecting the return radiation comprises a charged coupled device sensor array.

8. The device of claim 1, operable to use continuous wave or pulsed radiation for performing the optical scanning and further comprising means (124) for a time resolved acquisition of pulse shapes of pulses of primary and/or secondary radiation.

9. The device of claim 1, further comprising detector means (134, 136, 154, 156, 158, 170, 172, 174, 176; 196, 198, 198′, 200, 200′, 202) for detecting primary and/or secondary radiation, the secondary radiation having a different frequency than the primary radiation.

10. The device of claim 1, the means for optical scanning being adapted to perform the optical scanning from two opposite directions.

11. The device claim 1, the means for optically scanning comprising at least one component that is rotatably mounted for performing the acquisition of the imaging data from at least two different directions.

12. The device of claim 1, further comprising means for compressing the turbid medium.

13. The device of claim 1 being a scanning laser-pulse mammograph.

14. A method of imaging a turbid medium (130, 132; 184) comprising:
detecting an outer contour of the turbid medium,
optically scanning a sub-area of a maximum scanable area, wherein the sub-area is smaller than the maximum scanable area and covers the outer contour.

15. The method of claim 14, further comprising detecting radiation that is returned from the turbid medium in response to the optical scanning in a reverse direction.

16. The method of claim 14, wherein the contour of the turbid medium is detected by taking a picture using a charge coupled device camera.

17. The method of claim 16, wherein the charge coupled device camera is used for detecting the transmitted and/or return radiation.

18. The method of claim 14, wherein pulsed radiation is used for the optical scanning and further comprising time-resolved acquisition of the pulse shapes of the return radiation and/or transmitted radiation.

19. The method of claim 1, wherein the optical scanning is performed from two different directions.

20. The method of claim 1, wherein primary and secondary radiation is detected.

21. A computer program product comprising executable instructions for:
detecting an outer contour of a turbid medium,
controlling an optical scanner for optically scanning a sub-area of a maximum scanable area, wherein the sub-area is smaller than the maximum scanable area and covers the outer contour.
